(19) United States
(12) Patent Application Publication
(10) Pub. No.: US 2004/0229354 A1
(43) Pub. Date: Nov. 18, 2004

(54) Enhancement of humoral immune responses using a novel myeloid accessory cell

(76) Inventors: John Cambier, Denver, CO (US); David Mills, Sun City, CA (US); Michael Jordan, Denver, CO (US); Phiipa Marrack, Denver, CO (US)

Correspondence Address: SHERIDAN ROSS PC, 1560 BROADWAY, SUITE 1200, DENVER, CO, 80202

(21) Appl. No.: 10/760,115

(22) Filed: Jan. 16, 2004

Related U.S. Application Data
(60) Provisional application No. 60/440,842, filed on Jan. 16, 2003.

(51) Int. Cl. C12N 5/08

(52) U.S. Cl. 435/368

(57) ABSTRACT

An isolated myeloid cell, progenitors and progeny thereof, and populations of such cells are described. The cells have the phenotype of CD11b⁺, CD11c^{-/low}, MHC Class^{II-}, and mediate thymus-dependent immune responses, including IL-4 associated responses, and priming of B cells for MHC Class II signaling, expansion and proliferation. Also described are methods of making the cells and using the cells in therapeutic vaccines. Also described are methods of using the cells to identify agents, such as adjuvants, that are effective inducers of thymus-dependent immune responses, and particularly for priming of B cells.

### CROSS-REFERENCE TO RELATED APPLICATIONS

This application claims priority under 35 U.S.C. § 119(e) from U.S. Provisional Application Ser. No. 60/440,842, filed Jan. 16, 2003, entitled “Enhancement of Humoral Immune Response Using a Novel Myeloid Accessory Cell.” The entire disclosure of U.S. Provisional Application Ser. No. 60/440,842 is incorporated herein by reference.

### GOVERNMENT RIGHTS

This invention was made in part with government support under Grant Nos. AI-50802, AI-17134, AI-18785, AI-22295, AI-52225, and AI-20519, all awarded by the National Institutes of Health. The government may have certain rights to this invention.

### FIELD OF THE INVENTION

The present invention generally relates to a novel, isolated myeloid cell, wherein the cell enhances immune responses, and particularly, thymus-dependent immune responses, such as IL-4 associated immune responses, and more particularly, the priming of B cells for thymus-dependent immune responses (e.g., MHC Class 11-mediated signaling resulting in an immune response, expansion and/or antibody production).

### BACKGROUND OF THE INVENTION

Specific cell-cell interactions between T and B lymphocytes (also referred to as T or B cells) are required for B cell proliferation and differentiation during thymus-dependent antibody responses (1). During this cellular collaboration, contact-dependent signaling via distinct receptor-ligand pairs, including MHC class II and TCR, mediates reciprocal activation/differentiation of both cells (1). MHC class II aggregation delivers distinct signals to B cells of different activation states. Aggregation of class II on naive B cells induces cAMP generation and apoptosis (2,3). In contrast, aggregation of class II on IL-4 or antigen activated B cells leads to activation of tyrosine kinases, mobilization of intracellular free Ca²⁺, morphological changes, and proliferation (4-6). This difference is due to the fact that antigen and IL-4 induce the association of MHC class II molecules with signal-transducing Ig-a/β heterodimers (7). Induction of this change in signaling, which is referred to herein as “priming,” has not been shown to occur in vivo. Therefore, it would be of value to determine the means by which priming might be achieved in animals, and its possible significance for normal antibody responses.

### SUMMARY OF THE INVENTION

One embodiment of the present invention relates to an isolated myeloid cell and progenitors and progeny thereof, wherein the cell expresses CD11b, wherein the cell does not express MHC Class II, and wherein the cell expresses low levels of or does not express CD11c. In further aspects of the invention, the isolated myeloid cell has one or more of the following phenotypic characteristics: the cell expresses F4/80; the cell expresses CD68; the cell expresses CCR3; the cell expresses B220; the cell does not stain with vital red stain; the cell does not express CD86; the cell does not express a T cell receptor (TcR); and the cell does not express a surface immunoglobulin. In the embodiment where the cell is a murine cell, the cell expresses Gr1.

Preferably, the isolated cell of the present invention, when activated, mediates an immune response. In one aspect, the immune response is an immune response associated with IL-4 production. In one aspect, the isolated myeloid cell mediates priming of B cells for MHC class II signaling. In another aspect, the isolated myeloid cell mediates thymus-dependent B cell expansion. In another aspect, the isolated myeloid cell mediates thymus-dependent antibody production by B cells.

The isolated myeloid cell of the present invention, in one aspect, can be activated by an aluminum-based salt adjuvant. In another aspect, the isolated myeloid cell is activated by granulocyte-macrophage colony-stimulating factor (GM-CSF).

In one aspect of the invention, the isolated myeloid cell is derived from a cell isolated from bone marrow that has been exposed to granulocyte-macrophage colony-stimulating factor (GM-CSF). In another aspect, the cell is derived from a cell isolated from bone marrow that has been contacted with an aluminum-based salt adjuvant. In one embodiment, the isolated myeloid cell of the invention has been immortalized.

Another embodiment of the present invention relates to an isolated population of cells enriched for the isolated myeloid cell and progeny thereof as described above. In a preferred embodiment, the population is a clonal population consisting essentially of the myeloid cell or progenitors and progeny thereof as described above. In one embodiment, the population of cells is produced by: (a) isolating cells from a source selected from the group consisting of: bone marrow, hematopoietic precursor cells, adult stem cells, fetal stem cells, spleen cells, peripheral blood cells and embryonic stem cells; (b) exposing the cells to an agent selected from the group consisting of an aluminum-based salt adjuvant and GM-CSF, or a derivative thereof; and (c) isolating cells from step (b) that have the following cell surface phenotype: CD11b⁺, CD11c^{-/low}, MHC Class^{II-}. In another embodiment, the population of cells is produced by: (a) immunizing an animal with a composition comprising an aluminum-based salt adjuvant or a derivative thereof; and (b) isolating cells from step (a) that have the following cell surface phenotype: CD11b⁺, CD11c^{-/low}, MHC Class^{II-}.

Yet another embodiment of the present invention relates to a vaccine comprising the isolated myeloid cell or its progenitor as described above and at least one antigen. In one aspect, the antigen is selected from the group consisting of: a viral antigen, a mammalian cell surface molecule, a bacterial antigen, a fungal antigen, a protozoan antigen, a helminth antigen, an ectoparasite antigen, and a cancer antigen.

Another embodiment of the present invention relates to a vaccine comprising the isolated myeloid cell or its progenitor as described above and a cytokine.

Another embodiment of the present invention relates to a method for enhancing a thymus-dependent immune response, comprising: (a) isolating the myeloid cell or its progenitor as described above from a patient; (b) activating the cell ex vivo; and (c) administering the cell after step (b) to the patient. In one aspect, step (c) further comprises administering an antigen to the patient. In another aspect, step (b) comprises exposing the cell to an agent selected from the group consisting of an aluminum-based salt adjuvant and GM-CSF. In yet another aspect, the myeloid cell in (a) is isolated from the bone marrow, the spleen, or the peripheral blood of the patient.

Yet another embodiment of the present invention relates to a method for enhancing a thymus-dependent immune response, comprising: (a) providing a myeloid cell or its progenitor as described above; (b) activating the cell ex vivo; and (c) administering the cell after step (b) to the patient. In one aspect, step (c) further comprises administering an antigen to the patient. In another aspect, step (b) comprises exposing the cell to an agent selected from the group consisting of an aluminum-based salt adjuvant and GM-CSF.

Another embodiment of the present invention relates to a method to produce a myeloid cell that mediates thymus-dependent immune responses, comprising: (a) isolating cells from the bone marrow, spleen or peripheral blood of an animal; (b) exposing the cells to an agent selected from the group consisting of an aluminum-based salt adjuvant and GM-CSF, or a derivative thereof; and (c) selecting cells from (b) that have the following cell surface phenotype: CD11b⁺, CD11c^{-/low}, MHC Class^{II-}. In one aspect, the agent in step (b) is selected from the group consisting of an aluminum-based salt adjuvant and GM-CSF.

Yet another embodiment of the invention relates to a method to identify agents that enhance thymus-dependent immune responses, comprising: (a) exposing a source of myeloid progenitor cells to a test agent; (b) detecting whether cells from (a) that, after exposure to the test adjuvant, comprise cells having the following phenotype: CD11b⁺, CD11c^{-/low}, MHC Class^{II-}; and (c) determining whether cells detected in (b), when contacted with naive B cells, mediate priming of B cells for MHC class II signaling. An induction or increase in priming of B cells for MHC class II signaling when the bone marrow cells are exposed to the adjuvant indicates that the adjuvant is useful for enhancing thymus-dependent immune responses. In one aspect, step (a) is performed in vivo by administering the test adjuvant to an animal and isolating bone marrow cells, stem cells, or spleen cells from the animal prior to performing step (b). In this aspect, the test adjuvant can be administered together with an antigen. In another aspect, step (a) is performed in vitro by exposing the cells to the test adjuvant in a culture. In one aspect, the myeloid progenitor cells are selected from the group consisting of: bone marrow cells, adult stem cells, fetal stem cells, embryonic stem cells, hematopoietic precursor cells, spleen cells, peripheral blood cells, a direct progenitor of the myeloid cell as described above.

### BRIEF DESCRIPTION OF THE DRAWINGS OF THE INVENTION

FIG. 1A is a graph showing the effect of antigen/alum immunization on MHC class II/Ig-α/β signaling in splenic B cells.

FIG. 1B is a graph showing the percentage of B cells mobilizing intracellular Ca²⁺ following MHC class II aggregation following immunization with NP-CGG/alum.

FIG. 1C is a graph showing the effect of immunization with alum alone on MHC class II/Ig-α/β signaling in splenic B cells.

FIG. 1D is a graph showing a comparison of the effect on MHC class II/Ig-α/β signaling following immunization with NP-CGG/alum versus NP-CGG/CFA.

FIG. 2A is a graph showing MHC class II-mediated Ca²⁺ mobilization in splenic B220⁺ cells following exposure of mice to NP-B SA/alum with or without administration of depleting anti-Gr1⁺ antibody.

FIG. 2B is a graph showing MHC class II-mediated Ca²⁺ mobilization in B cells after coculture with Gr1⁺ or CD11c⁺ cells which were sorted from alum-injected or naïve mice.

FIG. 2C is a graph showing that Gr1⁺ cells and not CD11c⁺ cells, were selectively depleted after administration of depleting anti-Gr1⁺ antibody.

FIG. 2D is a graph showing MHC class II-mediated Ca²⁺ mobilization in B cells after culture of bone marrow Gr1⁺ cells with granulocyte-macrophage colony-stimulating factor (GM-CSF) and granulocyte colony-stimulating factor (G-CSF).

FIG. 3A is a graph showing MHC class II-mediated Ca²⁺ mobilization in naïve B cells from wild type or IL-4^{-/-} mice after culture with GM-CSF-activated bone marrow derived Gr1⁺ cells.

FIG. 3B is a graph showing MHC class II-mediated Ca²⁺ mobilization in naïve B cells from wild type or STAT6^{-/-} mice after culture with GM-CSF-activated bone marrow derived Gr1⁺ cells.

FIG. 3C is a graph showing MHC class II-mediated Ca²⁺ mobilization in B cells following culture with Gr1⁺ cells sorted from the spleens of NP-BSA/alum-immunized mice in the presence or absence of blocking anti-IL-4 antibodies.

FIG. 3D is a graph showing MHC class II-mediated Ca²⁺ mobilization in splenic B cells after exposure to NP-BSA/alum alone or in conjunction with blocking anti-IL-4 antibodies.

FIG. 4A is a graph showing production of NP-specific IgM antibody in mice vaccinated with NP-OVA/alum and subsequently treated with either anti-Gr1 or control antibodies.

FIG. 4B is a graph showing production of NP-specific IgG antibody in mice vaccinated with NP-OVA/alum and subsequently treated with either anti-Gr1 or control antibodies.

### DETAILED DESCRIPTION OF THE INVENTION

Exposure of naïve B cells to the cytokine, interleukin-4 (IL-4), and/or antigen leads to a state of “priming,” wherein subsequent aggregation of MHC class II induces Ca²⁺ mobilization and proliferation. According to the present invention, “priming”, with regard to B cells, refers to the induction of the association of MHC class II molecules with signal-transducing Ig-a/β heterodimers. However, prior to the present invention, it was not clear how critical this priming is for immune responses or how it is normally induced in vivo. The present inventors have discovered that injection of mice with the commonly used adjuvant, alum, leads to priming of splenic B cells, and to the accumulation in the spleen of a novel population of IL-4-producing myeloid cells, which in mice are Gr1⁺. These cells and IL-4 were required for in vivo priming of B cells, expansion of antigen-specific B cells, and optimal antibody production. The inventors' discovery reveals the novel role of an accessory myeloid population in the generation of immune responses, and particularly thymus-dependent humoral immune responses. The results described herein provide the first direct evidence that MHC class II/Ig-α/β signaling is primed in vivo during immune responses, and demonstrate that this occurs independently of antigen receptor specificity. They also show that a previously undescribed population of myeloid cells, which are Gr1⁺ in mice, accumulates in the spleens of alum-treated mice, mediates in vivo priming of B cells for MHC class II signaling, selectively produces or is otherwise associated with IL-4 after alum administration, and facilitates B cell immune responses. Accordingly, the present invention generally relates to a novel myeloid lineage cell that is useful for enhancing thymus-dependent immune responses and particularly, for priming naive B cells for activity in thymus-dependent immune responses, as well as uses of such cells in vaccines and other therapeutic methods. As discussed above, prior to the invention, the cells that were involved in in vivo priming of B cells for subsequent immune responses were not defined. However, the inventors have now discovered and characterized a myeloid lineage cell (described below), as well as its progenitor in bone marrow, and have shown that this cell primes B cells in vivo and in vitro. These cells can now be isolated from individual patients (e.g., from bone marrow, peripheral blood or spleen), or cell lines can be created from the myeloid lineage or its progenitor, and the cells can be used in novel vaccines (preventative and therapeutic) for priming of B cells and/or therapeutic methods to regulate diseases or conditions in which the priming of B cells for participation in an immune response would be beneficial (e.g., immunization in cancer and infectious disease, and vaccination against various pathogens or tumors). In addition, the novel myeloid cells of the present invention can be used as an indicator (end point) to test new adjuvants for the ability to induce or enhance cellular and humoral immune responses, and particularly, thymus-dependent immune responses, and in one aspect, B cell priming of thymus-dependent immune responses.

More specifically, the present inventors have defined a novel myeloid lineage cell (referred to herein as myeloid cells or myeloid accessory cells), which when stimulated with granulocyte-macrophage colony-stimulating factor (GM-CSF) and/or an adjuvant such as alum, functions to, at a minimum, prime naive B cells for participation in thymus-dependent immune responses and/or IL-4 associated immune responses. In one embodiment, the myeloid cell also primes IL-2- or other cytokine-associated immune responses. Progenitors of this cell have been identified by the present inventors in the bone marrow, and their functions can be activated by culture with GM-CSF. Alum injection of mice induces increased accumulation of these cells in the spleen and presumably other peripheral lymphoid organs where immune responses are initiated. This accessory cell has at least the following phenotype, as indicated by expression of cell surface markers: CD11b⁺, CD11c^{-/low}, MHC class II⁻. In addition, the murine cell expresses Gr1, and, without being bound by theory, the present inventors believe that the human equivalent may express a homologue of the murine Gr1. The novel myeloid cell of the present invention can also have the following phenotype: F4/80⁺, CD86⁻, CD68⁺, CCR3⁺, B220⁺, T cell receptor (TcR)⁻, and surface immunoglobulin (Ig)⁻. In addition, the novel myeloid cell of the present invention does not stain with vital red stain, a conventional eosinophil stain (trisodium salt of a sulfonated diazo dye (a ditolyl group diazotised to sulfonated aminonaphthalene residues), used as a vital stain).

In one embodiment, the clinical potential of this invention is as follows. The defined myeloid cell or its non-activated progenitor can be isolated from peripheral blood of subjects, isolated from the spleen of subjects, isolated from the bone marrow of subjects, or cultured as a cell line or immortalized cell line, primed or differentiated using GM-CSF or an aluminum-based salt, or other functional equivalent, and in some embodiments, an antigen, and then re-implanted/implanted in the individual, where an enhanced immune response, including an enhanced antibody response, is to occur. This approach is useful in vaccination of, for example, cancer patients, to increase tumor specific humoral immunity.

In another embodiment of the invention, this cell, its progenitor, and cell lines derived therefrom can be used as an indicator for testing new or known adjuvants for efficacy in stimulating or enhancing thymus-dependent immune responses, and particularly those associated with IL-4 production and B cell priming.

Accordingly, one embodiment of the present invention relates to an isolated myeloid cell and progenitors and progeny thereof, wherein the isolated myeloid cell has the following phenotype, as defined by cell surface markers: expresses CD11b (i.e., is CD11b⁺), does not express MHC Class II (i.e., is MHC Class II⁻), and expresses low levels of or does not express CD11c (i.e., is CD11c^{-/low}). The cell can also have the following additional phenotype: expresses F4/80 (F4/80⁺), expresses CD68 (CD68⁺), expresses CCR3 (CCR3⁺), expresses B220⁺, does not stain with vital red stain, does not express CD86 (CD86⁻), does not express a T cell receptor (TcR⁻), and does not express a surface immunoglobulin (mIg⁻). If the cell is a murine cell, then the cell expresses Gr1 (Gr1⁺).

As used herein, a “progenitor” cell refers to an ancestor of a cell (i.e. a cell from which a subject cell is derived). A myeloid cell of the present invention can be derived from a bone marrow cell, embryonic stem cell, adult stem cell, fetal cell, or hematopoietic precursor cell. The myeloid cell of the present invention can also be derived from or directly isolated from a spleen cell or peripheral blood cell. Preferably, the progenitor of a myeloid cell encompassed by the invention is the direct progenitor of the myeloid cell and represents a cell that has differentiated from the bone marrow or stem cell stage to a cell that, when appropriately stimulated (such as with an adjuvant or cytokine as described herein), can differentiate into the myeloid cell of the invention, preferably in an activated state. A “direct” progenitor is the cell that is one or two differentiation states higher than the myeloid cell of the invention, or can include the unactivated or naive form of the myeloid cell of the present invention. In other words, a direct progenitor when exposed to activating conditions, will differentiate or become activated to result in the myeloid cell of the invention which has the functional capabilities and the phenotype as described herein. A progenitor of the myeloid cell encompassed by the invention is a cell that is derived from one of these cell types described above (e.g., bone marrow, stem cells, spleen cells, peripheral blood cells) which, in murine cells expresses Gr1 (described below) and which, in cells from other species can express the homologue of Gr1. Further, the progenitor, when exposed to an aluminum-based salt or derivative thereof, GM-CSF or a derivative thereof, or another compound that has the same biological effect with regard to the differentiation of the myeloid cell, differentiates into the active myeloid cell of the present invention as described above. Methods to produce a myeloid cell of the present invention from starting cells such as bone marrow cells and thereby target the progenitor are described below, including in the Examples section.

In one embodiment, the progenitor cell (direct progenitor) of the myeloid cell of the invention (i.e., one or two of the closest or most proximal progenitors) has the phenotype: CD11b⁺, CD11c^{-/low}, MHC Class^{II-}. In another embodiment, the direct progenitor cell of the myeloid cell of the present invention has one or more of the following additional phenotypic characteristics: F4/80⁺, CD68⁺, CCR3⁺, B220⁺, does not stain with vital red stain, CD86⁻, TcR⁻, and mIg⁻. In one aspect, if the cell is a murine cell, then the cell is also Gr1⁺. In one aspect, if the cell is a human cell, it expresses the human homologue of Gr1. In yet another embodiment, the direct progenitor cell of the myeloid cell of the present invention has all of the following phenotypic characteristics: CD11b⁺, MHC Class II⁻, CD11c^{-/low}, F4/80⁺, CD68⁺, CCR3⁺, B220⁺, does not stain with vital red stain, CD86⁻, TcR⁻, and mIg⁻. In one aspect the cell also expresses Gr1 or the non-murine equivalent (homologue) thereof.

As used herein, a “progeny” cell refers to a cell derived from a subject cell, such as daughter cells resulting from cell division, as well as clones and cell lines derived from a subject cell.

A derivative of alum includes any aluminum-based salt that can be used as an adjuvant and particularly, that is capable of activating the myeloid cells of the present invention. A derivative of GM-CSF includes any modified form (homologue or analog) of GM-CSF with substantially similar biological activity of wild-type GM-CSF. According to the present invention, a homologue refers to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. An analog refers to a non-peptide compound that is able to mimic the biological action of a naturally occurring peptide, often because the mimetic has a basic structure that mimics the basic structure of the naturally occurring peptide and/or has the salient biological properties of the naturally occurring peptide. Various methods of drug design, useful to design or select analogs are disclosed in Maulik et al., 1997, Molecular Biotechnology: Therapeutic Applications and Strategies, Wiley-Liss, Inc., which is incorporated herein by reference in its entirety.

One characteristic of the isolated cell of present invention is that it is a myeloid cell. According to the present invention, a myeloid cell is defined as a cell of one of the lineages of non-lymphocytic, bone marrow-derived cells, including erythrocytes, platelets, megakaryocytes, granulocytes (neutrophils, eosinophils, basophils), dendritic cells, monocytes, macrophages, and mast cells. Myeloid cells are typically identified in the art by morphological analysis and cell surface marker identification, as well as by biological activity. Myeloid cells of the invention can be isolated from any member of the Vertebrate class, Mammalia, including, without limitation, primates, rodents, livestock and domestic pets.

As used herein, a cell surface marker refers to any compound on the surface of a cell that is detectable by techniques such as antibody binding studies, gel electrophoresis and various chromatography techniques known to those of skill in the art. A cell surface marker can include cell surface receptors, adhesion proteins, cell surface carbohydrate moieties, membrane-bound ligands and other molecules involved in cell to cell communication. Cell surface markers can be identified by several conventional techniques, many of which include using an antibody or other binding protein and one or more detectable markers. Techniques useful for determining protein expression include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry.

Antibodies that selectively bind to the cell surface markers described herein are known in the art and detection techniques for many of the markers described herein are discussed in the Examples section. The phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art as discussed above. Antibodies useful in detection methods can include polyclonal and monoclonal antibodies, divalent and monovalent antibodies, bi- or multi-specific antibodies, serum containing such antibodies, antibodies that have been purified to varying degrees, and any functional equivalents of whole antibodies (e.g., antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies)). The binding of an antibody or other antigen binding fragment can be detected through the use of any composition or label detectable by spectroscopic, photochemical, biochemical, immunochemical, electrical, optical or chemical means, including, but not limited to, biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

As discussed above, in a preferred embodiment, a myeloid cell of the present invention can have the following phenotype, as denoted by expression (or lack of expression) of various cell surface markers: CD11b⁺, MHC Class II⁻, CD11c^{-/low}, F4/80⁺, CD68⁺, CCR3⁺, B220⁺, does not stain with vital red stain, CD86⁻, TcR⁻, and mIg⁻. If the cell is a murine cell, then the cell is also Gr1⁺. In one aspect, if the cell is a human cell, it expresses the human homologue of Gr1.

According to the present invention, a cell is positive for the expression of a cell surface marker if the marker is detectable using conventional detection reagents and techniques at a level of at least about 2 fold above a background control (e.g., with a statistical significance of p<0.05). A cell that is positive for expression of a cell surface marker is typically a moderate or high expresser of the marker, and is denoted by Marker⁺, wherein “Marker” represents the name of the cell surface marker and “+” indicates a positive expression of the marker. A cell is a lower expresser of a cell surface marker if the marker is detectable at least about 2 fold over background but below about 10 fold over background. Such a cell marker can be denoted Marker^{+/-}. A cell marker can be denoted Marker^{low}, or Marker^{-/low}, if expression is either very low (e.g., about 2 fold above background or slightly lower than 2 fold above background) or sometimes not statistically significantly detectable at all using conventional detection techniques. A cell is negative for the expression of a cell surface marker, or Marker⁻, if substantially no marker is detected above background control (e.g., with a statistical significance of p<0.05).

CD11b (Protein Database AccessionNo. P11215; see also Corbi et al., J. Biol. Chem. 263 (25), 12403-12411 (1988)) is a type I transmembrane protein, also known in the art as αM integrin chain, αM-β2, C3biR, CR3, Mac-1, or Mol. Among cells of the myeloid lineage, CD11b is expressed by granulocytes and monocytes. Cd11b is implicated in various adhesive interactions of monocytes, macrophages and granulocytes as well as in mediating the uptake of complement-coated particles. It is identical with CR-3, the receptor for the iC3b fragment of the third complement component. It probably recognizes the r-g-d peptide in C3b. CD11b is also a receptor for fibrinogen, Factor X and ICAM 1. It recognizes p1 and p2 peptides of fibrinogen gamma chain.

CD11c (Protein Database Accession No. P20702; see also Corbi et al., EMBO J. 6 (13), 4023-4028 (1987)) is a type I transmembrane protein, also known in the art as αX integrin chain, Axb2, CR4, and leukocyte surface antigen p150,95. Among cells of the myeloid lineage, it is highly expressed on monocytes and macrophages, and moderately expressed on granulocytes. CD11c has been reported to have similar functions to CD11b/CD18 with which it cooperates, and is the CD11 component of the major CD11/CD18 receptor on tissue macrophages. CD11c is a receptor for fibrinogen, and recognizes the sequence g-p-r in fibrinogen. It mediates cell-cell interaction during inflammatory responses, and is especially important in monocyte adhesion and chemotaxis.

CD68 (Protein Database Accession No. P34810; see also Holness et al., Blood 81 (6), 1607-1613 (1993)), also known in the art as gp110 and macrosialin, can be expressed in least small amounts on the surface of many myeloid cells including, but it is highly expressed by blood monocytes and tissue and is considered to be a marker for cells of the monocyte/macrophage lineage. CD68 could play a role in phagocytic activities of tissue macrophages, both in intracellular lysosomal metabolism and extracellular cell-cell and cell-pathogen interactions. CD68 binds to tissue- and organ-specific lectins or selectins, allowing homing of macrophage subsets to particular sites. Rapid recirculation of CD68 from endosomes, lysosomes to the plasma membrane may allow macrophages to crawl over selectin bearing substrates or other cells.

F4/80 (Protein Database Accession No. NP_{—}034260; see also McKnight et al., J. Biol. Chem. 271 (1), 486-489 (1996)) is a murine membrane protein, also known in the art as EGF-like module containing, mucin-like, hormone receptor-like sequence 1, and lymphocyte antigen 71. Among myeloid cells, F4/80 is expressed by murine macrophages and blood monocytes. It has an epidermal growth factor (EGF)-like domain and a G-protein coupled transmembrane domain. Human epidermal growth factor (EGF) module-containing mucin-like hormone receptor 1 (EMR1; Protein Database Accession No. NP_{—}001965; see also Baud et al., Genomics 26 (2), 334-344 (1995)) is the predicted human homologue of F4/80. EMR1 has a domain resembling seven transmembrane G protein-coupled hormone receptors (7TM receptors) at its C-terminus. The N-terminus of the encoded protein has six EGF-like modules, separated from the transmembrane segments by a serine/threonine-rich domain, a feature reminiscent of mucin-like, single-span, integral membrane glycoproteins with adhesive properties.

B220 (Protein Database Accession No. P08575; see also Streuli et al., J. Exp. Med. 166 (5), 1548-1566 (1987)) is a type I membrane protein, also known in the art as CD45, CD45R, EC 3.1.3.4, Leukocyte Common Antigen (LCA), T200, and Ly5. B220 is a tyrosine phosphatase with critical requirement for T and B cell antigen receptor-mediated activation. The first PTPAse domain has enzymatic activity, while the second one seems to affect the substrate specificity of the first. B220 is typically expressed on many hematopoietic cells.

CCR3 (Protein Database Accession No. NP_{—}003956; see also Fan et al., Biochem. Biophys. Res. Commun. 243 (1),264-268 (1998)), is also known as chemokine (C-C motif) receptor-like 2. CCR3 is a chemokine receptor like protein, which is predicted to be a seven transmembrane protein and most closely related to CCR1. Chemokines and their receptors mediate signal transduction that is critical for the recruitment of effector immune cells to the site of inflammation. This gene is expressed at high levels in primary neutrophils and primary monocytes, and is further upregulated on neutrophil activation and during monocyte to macrophage differentiation.

CD86 (Protein Database Accession No. P42081; see also Freeman et al., Science 262 (5135), 909-911 (1993) is a type I membrane protein, also known as B7-2 and B70. CD86 is the receptor involved in the costimulatory signal essential for T lymphocyte proliferation and interleukin-2 (IL-2) production, by its binding of CD28 or CTLA-4. CD86 may play a critical role in the early events of T cell activation and costimulation of naive T cells. Among myeloid cells, CD86 is expressed constitutively by interdigitating dendritic cells in T zones of secondary lymphoid organs and at lower levels by Langerhans cells and peripheral blood dendritic cells. It has also been reported to be expressed at low levels by monocytes.

Gr1 is a murine protein also known as Ly-6G (Protein Database Accession No. P35461; see also Fleming et al., J. Immunol. 150 (12), 5379-5390 (1993)). A GPI-linked protein, Gr-1 is expressed by the myeloid lineage in a developmentally regulated manner in the bone marrow. While monocytes only express Gr-1 transiently during their bone marrow development, the expression of Gr-1 on bone marrow granulocytes as well as on peripheral neutrophils is a good marker for these populations. The homologue or equivalent of Gr1 in non-murine cells can be identified using conventional molecular techniques. For example, one can generate or obtain a cDNA or genomic DNA library prepared from myeloid cells from the organism of interest and use degenerate oligonucleotide primers designed using amino acid motifs or sequences from Gr1, for example to obtain a nucleic acid probe molecule to screen genomic or cDNA libraries; one might also use genetic approaches based on protein-protein interactions (e.g. a yeast two-hybrid system). One may also use one of a variety of publicly available bioinformatics resources to screen sequence databases for homologues of murine Gr1.

Major histocompatibility (MHC) proteins are generally classified into two categories: class I and class II MHC proteins. An MHC class I protein is an integral membrane protein comprising a glycoprotein heavy chain, also referred to herein as the a chain, which has three extracellular domains (i.e., α₁, α₂ and α₃) and two intracellular domains (i.e., a transmembrane domain (TM) and a cytoplasmic domain (CYT)). The heavy chain is noncovalently associated with a soluble subunit called β2-microglobulin (β2m). An MHC class II protein is a heterodimeric integral membrane protein comprising one α chain and one β chain in noncovalent association. The α chain has two extracellular domains (α₁ and α₂), and two intracellular domains (a TM domain and a CYT domain). The β chain contains two extracellular domains (β₁ and β₂), and two intracellular domains (a TM domain and CYT domain). Many human and other mammalian MHC molecules are well known in the art. MHC proteins. Among myeloid cells, MHC Class II is expressed by monocytes, macrophages and dendritic cells. MHC Class II proteins typically present antigenic peptides derived either from exogenous proteins that enter a cell's endocytic pathway or from proteins synthesized in the ER. Intracellular trafficking permits an antigenic peptide to become associated with an MHC protein. The resulting MHC-peptide complex then travels to the surface of the cell where it is available for interaction with a T cell receptor (TcR).

A T cell receptor (TcR) according to the present invention is the T cell antigen receptor that is expressed on the surface of T lymphocytes (T cells). The TcR is composed of an α and a β chain (TCR-α/β) or a γ and a δ chain (TcR-γ/δ), and is associated on T cells with the CD3 complex, composed of γ, δ, and ε and dimers of the TCR-ζ family proteins (ζ and η).

Surface immunoglobulin (Ig), or membrane Ig (mlg), is noncovalently associated with heterodimers of Ig-α and Ig-β to form B cell antigen receptors on B lymphocytes (B cells).

An isolated myeloid cell of the present invention also has functional characteristics (biological activities) that define the cell and that are important to the ultimate utility of the cell. In general, the biological activity or biological action of a cell or protein refers to any function(s) exhibited or performed by the cell or protein that is ascribed to the naturally occurring form of the cell or protein as measured or observed in vivo (i.e., in the natural physiological environment of the cell or protein) or in vitro (i.e., under laboratory conditions). The isolated myeloid cell of the present invention, when activated, has been shown to be capable of mediating an immune response, and particularly, a thymus-dependent immune response. A “thymus-dependent immune response” is a cell-mediated immune response that depends on the involvement of T lymphocytes. B lymphocytes (and other antigen presenting cells) participate in thymus-dependent immune responses by providing important interactions with and signals for T lymphocytes in the form of antigen presentation and cytokine secretion, for example. In turn, T lymphocytes and associated cells provide signals and secrete factors that induce B lymphocyte differentiation and proliferation, and lead to thymus-dependent antibody production by the B lymphocyte.

The present inventors have also discovered that the myeloid cells of the present invention, when activated, are particularly effective at mediating an immune response associated with interleukin-4 (IL-4) activity. This biological activity is mediated at a minimum by association of the myeloid cell with the presence of IL-4 during the immune response, and in one embodiment, the myeloid cell may actually either carry or produce IL-4 in the region of the immune response. IL-4 is a cytokine produced by some T lymphocytes and various myeloid cells (e.g., eosinophils, mast cells, basophils). IL-4 exerts different effects on B cells at different stages in the cell cycle. On resting B-cells, IL-4 acts as an activating factor, inducing them to enlarge in size and increase MHC Class II expression. Following activation by an antigen or mitogen, IL-4 acts as a growth factor, driving DNA replication in the B cells. In the case of proliferating B cells, IL-4 acts as a differentiation factor by regulating isotype class switching. IL-4 also plays a major role in T cell development. It is influential in promoting differentiation of T helper cells into TH2 cells during an immune response, and can also act as a mast cell growth factor. In further embodiments of the invention, the myeloid cells of the invention, when activated, are particularly effective at mediating an immune response associated with another cytokine that is involved in thymus-dependent immune responses. For example, in humans, such a cytokine can include IL-2, IL-4 or other cytokines. In one aspect, the immune response associated with another cytokine that performs the equivalent function of IL-4 in the murine system as described herein (see the Examples section) is enhanced by the myeloid cell of the present invention.

A particularly interesting functional characteristic of a myeloid cell of the invention is the ability of the cell, when activated, to mediates priming of B cells for MHC class II signaling, B cell expansion, and antibody production by B cells. “Priming” of B cells, according to the present invention, refers to the induction of the association of MHC class II molecules with signal-transducing Ig-a/β heterodimers, which enables the B cell to receive signals that result in activation of the B cell. B cell expansion occurs upon activation of B cells and can include B cell proliferation and clonal expansion. After extensive proliferation and clonal expansion, B cells can differentiate into plasma cells that produce and secrete antibodies.

According to the present invention, activation of a cell occurs when the cell is exposed to a triggering, stimulating or inducing event that causes the cell to undergo one or more changes or reactions that result in the ability of the cell to perform one or more biological functions or that result in the execution of one or more biological functions by the cell. For example, a triggering, stimulating or inducing event can include, but is not limited, to exposure to one or more biological response modifiers (e.g., adjuvants, cytokines, hormones, chemicals, soluble proteins, etc.), or contact with another cell (e.g., a receptor/ligand interaction or other protein/protein interaction). Activation of a cell typically results in various signal transduction events (e.g., changes in expression or location of intracellular signaling molecules, phosphorylation of proteins, dephosphorylation of proteins, conformational changes in proteins, association of proteins, dissociation of proteins, etc.) that can result in a variety of responses, including cytoskeletal reorganization, calcium mobilization, proliferation of a cell, differentiation of a cell, changes (up- or downregulation) in the expression of cell surface markers, up-or downregulation of secretion of products by the cell (e.g., hormones, cytokines, etc.), changes in migration of the cell, etc. A myeloid cell of the present invention is activated cell upon exposure to (e.g., contact with) an agent that results in a myeloid cell having the phenotypic characteristics discussed above, which is now capable of mediating immune responses in vitro or in vivo, such immune responses having been discussed above. The present inventors have found that the known adjuvant, alum, and the known cytokine, granulocyte-macrophage colony-stimulating factor (GM-CSF) are both capable of producing activated myeloid cells of the present invention. For example, immunization of an animal with alum (alone or in conjunction with an antigen), results in the presence of the myeloid cells of the invention in at least the spleen of the animal, such cells now being capable of mediating immune responses, and particularly, IL-4 associated immune responses, such as the priming of B cells for MHC Class II signaling, expansion and antibody production. Without being bound by theory, the present inventors believe that the myeloid cell of the invention may also prime other aspects of thymus-dependent immunity, such as through effects on T cell activity and the activity of other immune cells. In another example, exposure of bone marrow cells to GM-CSF results in the appearance/activation of a myeloid cells of the invention that are capable of mediating immune responses as described herein. The present invention encompasses any means of inducing the appearance, mobilization, differentiation of and/or activation of the myeloid cells of the invention, such as by stimulation with other adjuvants, compounds, or cytokines. Cells may also be isolated from spleen or peripheral blood for use in these methods. Indeed, the cells of the invention can be used in assays to identify other adjuvants, cytokines and compounds that provide this effect, as such agent will be particularly useful in the enhancement of thymus-dependent immune responses in an individual.

According to the present invention, an “isolated” myeloid cell is a cell that has been removed from its natural milieu (i.e., that has been subject to human manipulation) and that is preferably provided in a form that is enriched for that cell as compared to the relative presence of that cell in the natural milieu. Isolated cells can therefore include purified myeloid cells as well as partially purified cells or cell populations that have been enriched for the myeloid cell. As such, “isolated” does not necessarily reflect the extent to which the protein has been purified. Isolated cells of the present invention are, in one embodiment, retrieved, obtained, and/or used in “substantially pure” form. As used herein, “substantially pure” refers to a purity that allows for the effective use of the cell in vitro, ex vivo or in vivo according to any methods or utilities described for the present invention. For a cell to be useful in an in vitro, ex vivo or in vivo method according to the present invention, it is usually substantially free of contaminants, other cells and/or chemicals that might interfere or that would interfere with its use in a method disclosed by the present invention, or that at least would be undesirable for inclusion with the cell when it is used in a method disclosed by the present invention.

One embodiment of the invention relates to an isolated population of cells that is enriched for the myeloid cell of the invention. A population of cells that is “enriched” for a given cell type refers to a population of cells that have been exposed to some process or condition (e.g., positive or negative selection, culture conditions that induce the growth of the cell, etc.) that results in a relative increase in the number of the given cell type as a percentage of the total number of cells in the population. Methods for enriching cells in a cell population are well known in the art and are readily useful on the myeloid cells of the invention, particularly given the identification of the cell surface markers expressed (and not expressed) by the cell. Such markers can be used in positive and/or negative selection strategies to enrich for the myeloid cells of the invention. Similarly, exposure of a culture of, for example, bone marrow cells, to an agent such as GM-CSF has also been demonstrated to be an effective means of enriching for the presence of the myeloid cell of the invention. Immunization of an animal with alum, for example, will also result in an enrichment of the myeloid cells of the invention in the spleen, whereby such cells can be isolated from the spleen using the phenotypic markers as described herein. Preferably, an enrichment protocol results in a population that is enriched by at least about 5% for the myeloid cells of the invention as compared to prior to the enrichment protocol (i.e., the total number of myeloid cells of the invention as a percentage of the total number of cells in the population increases by 5% after the enrichment process). More preferably, the enrichment results in at least a 10% enrichment for the myeloid cells, and more preferably, at least about 15%, and more preferably, at least about 20%, and so on, in increments of 5% (25%, 30%, 35%) to an enrichment of 100%, 200%, 300% and higher (10×, 20×, 50×, 100×). Preferably, an enriched population of myeloid cells of the invention comprises at least about 25% of the total cells as the myeloid cells of the invention, and more preferably at least about 30%, and more preferably at least about 35%, and so on, in increments of 5% (40%, 45%, 50%) to an enrichment of up to 100% myeloid cells of the invention as a percentage of the total number of cells in the population. An enriched population of myeloid cells can include close progenitors and progeny of the myeloid cells.

Preferably, a “substantially pure” cell population, as referenced herein, is a population of cells that has been purified or enriched for a single, desired cell type such that the desired cell type comprises at least about 80% weight/weight of the total cells in a given population of cells, and more preferably, at least about 85%, and more preferably at least about 90%, and more preferably at least about 91%, and more preferably at least about 92%, and more preferably at least about 93%, and more preferably at least about 94%, and more preferably at least about 95%, and more preferably at least about 96%, and more preferably at least about 97%, and more preferably at least about 98%, arid more preferably at least about 99%, weight/weight of the total cells in a given population of cells. A population “consisting essentially of” a given cell type refers to a population of cells in which the given cell type comprises at least about 95% of the total cells in the population, and more preferably about 96% of the total cells in the population, and more preferably about 97% of the total cells in the population, and more preferably about 98% of the total cells in the population, and more preferably about 99% of the total cells in the population, and more preferably greater than about 99% of the total cells in the population. A purified population of myeloid cells can include direct progenitors (i.e., proximal, close, immediate progenitors) and progeny of the myeloid cell.

According to the present invention, a population of the novel myeloid cells of the present invention is preferably at least about 70% clonal, more preferably at least about 75% clonal, more preferably at least about 80% clonal, more preferably at least about 85% clonal, more preferably at least about 90% clonal, and even more preferably at least about 95% clonal. As used herein, the term “clonal” refers to a group of cells that are of a single cell type (e.g., that all have essentially the same phenotype, that all express the same surface markers or display essentially the same responsiveness to a growth factor).

The isolated population of myeloid cells of the present invention can be produced by several suitable methods. For example, one can: (a) isolate cells from a source selected from: bone marrow, hematopoietic precursor cells, adult stem cells, fetal stem cells, and embryonic stem cells; (b) expose the cells an agent that is known to activate the myeloid cells of the invention (e.g., alum or GM-CSF, or a derivative thereof); and isolate cells from step (b) that have the following cell surface phenotype: CD11b⁺, CD11c^{-/low}, MHC Class^{II-}. One can further test the cells to confirm that they have the desired functional activity if desired, and one can also test for the presence or absence of other cell surface markers as discussed herein. As another example, one can produce a population cells enriched for the myeloid cells of the invention by: (a) immunizing an animal with a composition comprising alum or a derivative thereof; (b) isolating cells from the animal after step (a) that have the following cell surface phenotype: CD11b⁺, CD11c^{-/low}, MHC Class^{II-}. Again, one can further test the cells to confirm that they have the desired functional activity if desired, and one can also test for the presence or absence of other cell surface markers as discussed herein.

To produce, culture, or differentiate a myeloid cell according to the present invention, one can start with a cell population that contains progenitors for the myeloid cell. Such cell populations include, but are not limited to, cells expanded from any source of hematopoietic stem cells, such as a hematopoietic stem cell population or precursor population that contains myeloid precursors, bone marrow, fetal and adult stem cells, and embryonic stem cells, which include germ cells and embryo-derived stem cells, spleen cells, and peripheral blood cells. In order to provide an expanded population containing myeloid progenitors, a starting cell (e.g., a stem cell) is typically cultured under conditions which expand and differentiate the hematopoietic precursors to a myeloid cell precursor cell phenotype. Any pool of progenitors as described herein can be differentiated to a point where the direct progenitor cell population for the myeloid cell of the present invention will generate myeloid cells of the invention when exposed to the preferred agents as described herein (e.g., alum, GM-CSF, derivatives thereof or other agents having the same biological property). In one aspect of the invention, stem cells, such as embryonic stem cells, are cultured under conditions suitable to expand the hematopoietic stem cells, such as in a liquid culture or semi-solid culture, in the absence of stromal cells.

One method for producing an expanded culture of hematopoietic progenitors is described in U.S. Pat. No. 5,914,268 and in U.S. Pat. No. 5,874,301, each of which is incorporated herein by reference in its entirety. The present invention is not limited to this method of expansion; other methods of producing hematopoietic/myeloid progenitor cells are also encompassed by the present invention. Other methods of producing expanded populations of hematopoietic precursors are described, for example, in Nakano et al., 1995, Seminars in Immunology 7(3):197-203; U.S. Pat. No. 5,646,043 to Emerson et al.; U.S. Pat. No. 5,827,742 to Scadden et al.; U.S. Pat. No. 5,861,315 to Nakahata et al.; U.S. Pat. No. 6,440,734 to Pykett et al.; or U.S. Pat. No. 6,326,198 to Emerson et al., each of which is incorporated herein by reference in its entirety. In one embodiment, the cell population provided as a starting material to produce the myeloid cell of the invention is a hematopoietic precursor cell population that has been expanded from an embryonic stem cell population. For example, such a population includes the embryoid body cell population and precursor cell populations derived therefrom described in U.S. Pat. No. 5,914,268 and in U.S. Pat. No. 5,874,301, ibid.

As used herein, a step of “providing” a given cell refers to any means of beginning the next step in a method with the necessary starting population of cells, including by culturing a cell population to produce such cells, purchasing such cells, or obtaining such cells from a source laboratory.

An isolated myeloid cell of the present invention or direct progenitors or progeny thereof, or a population of cells enriched for the myeloid cell of the invention can be cultured in any available medium which has been developed for culture of animal cells and particularly, mammalian cells, or which can be prepared in the laboratory with the appropriate components necessary for animal cell growth, such as assimilable carbon, nitrogen and micronutrients. Such a medium comprises a base medium, which is any base medium suitable for animal cell growth, including, but not limited to, Iscove's Modified Dulbecco's Medium (IMDM), Dulbecco's modified Eagles medium (DMEM), alpha MEM (Gibco), RPMI 1640, or any other suitable commercially available media. To the base medium, assimilable sources of carbon, nitrogen and micro-nutrients are added including, but not limited to, a serum source, growth factors, amino acids, antibiotics, vitamins, reducing agents, and/or sugar sources. It is noted that completed mediums comprising a base medium and many of the additional components necessary for animal cell growth are commercially available, and some media are available for particular types of cell culture, such as the Myelocult M5300 medium from StemCell Technologies, which was developed and is commercially available for the long term culture of myeloid cells. Myelocult M5300 comprises: 12.5% horse serum, 12.5% fetal bovine serum, 0.2 mM I Inositol, 16 μm folic acid, 10⁻⁴ m 2-mercaptoethanol, 2 mM L-glutamine, and alpha MEM. Therefore, one can prepare or purchase a medium suitable for the culture of animal cells or more particularly, myeloid cells, and then further supplement the medium as necessary (e.g., by adding cytokines or another component).

In one embodiment of the present invention, the myeloid cell of the invention, or progenitors or progeny thereof, are immortalized for long term culture. Methods for immortalization of primary cells are known in the art. One effective method for cell immortalization is described, for example, in PCT Publication No. WO 00/43500, incorporated herein by reference in its entirety.

The novel myeloid cells of the invention can be used in a variety of basic and applied research applications, as well as for therapeutic treatment of patients, as described herein. For example, as discussed above, the cells of the present invention enable the systematic evaluation of various adjuvants and other compounds on the priming of immune responses and particularly, on the enhancement of thymus-dependent immune responses, IL-4 associated immune responses, and particularly on B cell priming of MHC Class II signaling, B cell expansion and B cell antibody production. The cells of the invention are also useful as vaccine components to enhance an immune response, either non-specifically or in conjunction with immunization with an antigen. Furthermore, scaled-up production of the myeloid cells with distinct features (i.e., genetically modified cells) can be generated using gene targeting, and such cells can be used for therapeutic purposes. Such therapeutic purposes include, but are not limited to, correcting myeloid cell deficiencies, correcting functional defects, and treating diseases in which enhancement of a thymus-dependent, IL-4 associated (or other cytokine with equivalent immune response-related activity) and/or B cell response is beneficial.

Accordingly, another embodiment of the invention relates to a vaccine comprising an isolated myeloid cell (and/or progenitors or progeny thereof) or population thereof of the present invention. In one embodiment, the isolated myeloid cell or population comprising such cell is administered alone or with a pharmaceutically acceptable excipient. In one embodiment, the isolated myeloid cell of the invention is formulated with at least one biological response modifier to enhance the immune response. According to the present invention, a biological response modifier is a compound that can modulate a biological response, and particularly an immune response. Certain biological response modifiers can stimulate a protective immune response whereas others can suppress a harmful immune response. Certain biological response modifiers preferentially enhance a cell-mediated immune response whereas others preferentially enhance a humoral immune response (i.e., can stimulate an immune response in which there is an increased level of cellular compared to humoral immunity, or vice versa.). There are a number of techniques known to those skilled in the art to measure stimulation or suppression of immune responses, as well as to differentiate cellular immune responses from humoral immune responses. Suitable biological response modifiers include cytokines, hormones, lipidic derivatives, small molecule drugs and other growth modulators, such as, but not limited to, interleukin 2 (IL-2), interleukin 4 (IL-4), interleukin 10 (IL-10), interleukin 12 (IL-12), interferon gamma (IFN-gamma) insulin-like growth factor I (IGF-I), transforming growth factor beta (TGF-β), steroids, prostaglandins and leukotrienes.

In another embodiment, the isolated myeloid cell of the invention (and/or progenitors or progeny thereof) is combined with or formulated for administration with at least one antigen. A biological response modifier as described above may also be included, if desired. The vaccine can include, one, two, a few, several or a plurality of antigens, including one or more immunogenic domains of one or more antigens, as desired. According to the present invention, the general use herein of the term “antigen” refers: to any portion of a protein (peptide, partial protein, full-length protein), wherein the protein is naturally occurring or synthetically derived, to a cellular composition (whole cell, cell lysate or disrupted cells), to an organism (whole organism, lysate or disrupted cells) or to a carbohydrate or other molecule, or a portion thereof, wherein the antigen elicits an antigen-specific immune response (humoral and/or cellular immune response), or alternatively acts as a toleragen, against the same or similar antigens that are encountered within the cells and tissues of the animal to which the antigen is administered.

In one embodiment of the present invention, when it is desirable to stimulate an immune response, the term “antigen” can be used interchangeably with the term “immunogen”, and is used herein to describe a protein, peptide, cellular composition, organism or other molecule which elicits a humoral and/or cellular immune response (i.e., is antigenic), such that administration of the immunogen to an animal (e.g., via a vaccine of the present invention) mounts an antigen-specific immune response against the same or similar antigens that are encountered within the tissues of the animal. Therefore, to vaccinate an animal against a particular antigen means, in one embodiment, that an immune response is elicited against the antigen as a result of administration of the antigen. Vaccination preferably results in a protective or therapeutic effect, wherein subsequent exposure to the antigen (or a source of the antigen) elicits an immune response against the antigen (or source) that reduces or prevents a disease or condition in the animal. The concept of vaccination is well known in the art. The immune response that is elicited by administration of a therapeutic composition of the present invention can be any detectable change in any facet of the immune response (e.g., cellular response, humoral response, cytokine production), as compared to in the absence of the administration of the vaccine.

In another embodiment, when it is desirable to suppress an immune response against a given antigen, an antigen can include a toleragen. According to the present invention, a toleragen is used to describe a protein, peptide, cellular composition, organism or other molecule that is provided in a form, amount, or route of administration such that there is a reduced or changed immune response to the antigen, and preferably substantial non-responsiveness, anergy, other inactivation, or deletion of immune system cells in response to contact with the toleragen or a cell expressing or presenting such toleragen.

A “vaccinating antigen” can be an immunogen or a toleragen, but is an antigen used in a vaccine, where a biological response (elicitation of an immune response, tolerance) is to be elicited against the vaccinating antigen.

An immunogenic domain of a given antigen can be any portion of the antigen (i.e., a peptide fragment or subunit) that contains at least one epitope that acts as an immunogen when administered to an animal. For example, a single protein can contain multiple different immunogenic domains.

An epitope is defined herein as a single immunogenic site within a given antigen that is sufficient to elicit an immune response, or a single toleragenic site within a given antigen that is sufficient to suppress, delete or render inactive an immune response. Those of skill in the art will recognize that T cell epitopes are different in size and composition from B cell epitopes, and that epitopes presented through the Class I MHC pathway differ from epitopes presented through the Class II MHC pathway. An antigen can be as small as a single epitope, or larger, and can include multiple epitopes. As such, the size of an antigen can be as small as about 5-12 amino acids (e.g., a peptide) and as large as: a full length protein, including a multimer and fusion proteins, chimeric proteins, whole cells, whole microorganisms, or portions thereof (e.g., lysates of whole cells or extracts of microorganisms). In addition, antigens include carbohydrates, such as those expressed on cancer cells. In preferred embodiments, the antigen is selected from the group of a tumor antigen or an antigen of an infectious disease pathogen (i.e., a pathogen antigen). In one embodiment, the antigen is selected from the group of: a viral antigen, an overexpressed mammalian cell surface molecule, a bacterial antigen, a fungal antigen, a protozoan antigen, a helminth antigen, an ectoparasite antigen, a cancer antigen, a mammalian cell molecule harboring one or more mutated amino acids, a protein normally expressed pre- or neo-natally by mammalian cells, a protein whose expression is induced by insertion of an epidemiologic agent (e.g. virus), a protein whose expression is induced by gene translocation, and a protein whose expression is induced by mutation of regulatory sequences.

According to the present invention, an antigen suitable for use in the present vaccine can include two or more immunogenic domains or epitopes from the same antigen, two or more antigens immunogenic domains, or epitopes from the same cell, tissue or organism, or two or more different antigens, immunogenic domains, or epitopes from different cells, tissues or organisms.

Tumor antigens useful in the present invention can include a tumor antigen such as a protein, glycoprotein or surface carbohydrates from a tumor cell, an epitope from a tumor antigen, an entire tumor cell, mixtures of tumor cells, and portions thereof (e.g., lysates). In one embodiment, tumor antigens useful in the present invention can be isolated or derived from an autologous tumor sample. An autologous tumor sample is derived from the animal to whom the therapeutic composition is to be administered. Therefore, such antigens will be present in the cancer against which an immune response is to be elicited. In one aspect, the tumor antigen provided in a vaccine is isolated or derived from at least two, and preferably from a plurality of allogeneic tumor samples of the same histological tumor type. According to the present invention, a plurality of allogeneic tumor samples are tumor samples of the same histological tumor type, isolated from two or more animals of the same species who differ genetically at least within the major histocompatibility complex (MHC), and typically at other genetic loci. Therefore, if administered together, the plurality of tumor antigens can be representative of substantially all of the tumor antigens present in any of the individuals from which antigen is derived. This embodiment of the method of the present invention provides a vaccine which compensates for natural variations between individual patients in the expression of tumor antigens from tumors of the same histological tumor type. Therefore, administration of this therapeutic composition is effective to elicit an immune response against a variety of tumor antigens such that the same therapeutic composition can be administered to a variety of different individuals. In some embodiments, antigens from tumors of different histological tumor types can be administered to an animal, in order to provide a very broad vaccine.

Preferably, the tumor from which the antigen is isolated or derived is any tumor or cancer, including, but not limited to, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, primary hepatic cancers, lung cancers, pancreatic cancers, gastrointestinal cancers, renal cell carcinomas, hematopoietic neoplasias and metastatic cancers thereof.

According to the present invention, a cancer antigen can include any tumor antigen as described above, in addition to any other antigen that is associated with the risk of acquiring or development of cancer or for which an immune response against such antigen can have a therapeutic benefit against a cancer. For example, a cancer antigen could include, but is not limited to, a tumor antigen, a mammalian cell molecule harboring one or more mutated amino acids, a protein normally expressed pre- or neo-natally by mammalian cells, a protein whose expression is induced by insertion of an epidemiologic agent (e.g. virus), a protein whose expression is induced by gene translocation, and a protein whose expression is induced by mutation of regulatory sequences. Some of these antigens may also serve as antigens in other types of diseases (e.g., autoimmune disease).

In one aspect of the invention, the antigen useful in the present composition is an antigen from a pathogen (including the whole pathogen), and particularly, from a pathogen that is associated with (e.g., causes or contributes to) an infectious disease. An antigen from an infectious disease pathogen can include antigens having epitopes that are recognized by T cells, antigens having epitopes that are recognized by B cells, antigens that are exclusively expressed by pathogens, and antigens that are expressed by pathogens and by other cells. Pathogen antigens can include whole cells and the entire pathogen organism, as well as lysates, extracts or other fractions thereof. In some instances, an antigen can include organisms or portions thereof which may not be ordinarily considered to be pathogenic in an animal, but against which immunization is nonetheless desired. The antigens can include one, two or a plurality of antigens that are representative of the substantially all of the antigens present in the infectious disease pathogen against which the vaccine is to be administered. In other embodiments, antigens from two or more different strains of the same pathogen or from different pathogens can be used to increase the therapeutic efficacy and/or efficiency of the vaccine.

According to the present invention, a pathogen antigen includes, but is not limited to, an antigen that is expressed by a bacterium, a virus, a parasite or a fungus. Preferred pathogen antigens for use in the method of the present invention include antigens which cause a chronic infectious disease in an animal. In one embodiment, a pathogen antigen for use in the method or composition of the present invention includes an antigen from a virus.

Other preferred antigens to include in compositions (vaccines) of the present invention include antigens that are capable of suppressing an undesired, or harmful, immune response, such as is caused, for example, by allergens, autoimmune antigens, inflammatory agents, antigens involved in GVHD, certain cancers, septic shock antigens, and antigens involved in transplantation rejection. Such compounds include, but are not limited to, antihistamines, cyclosporin, corticosteroids, FK506, peptides corresponding to T cell receptors involved in the production of a harmful immune response, Fas ligands (i.e., compounds that bind to the extracellular or the cytosolic domain of cellular Fas receptors, thereby inducing apoptosis), suitable MHC complexes presented in such a way as to effect tolerization or anergy, T cell receptors, and autoimmune antigens, preferably in combination with a biological response modifier capable of enhancing or suppressing cellular and/or humoral immunity.

Other antigens useful in the present invention and combinations of antigens will be apparent to those of skill in the art. The present invention is not restricted to the use of the antigens as described above.

A vaccine comprising a myeloid cell of the invention (and/or progenitors or progeny thereof) contains from about 0.5×10⁶ to about 5.5×10¹⁰ myeloid cells per single dose per individual patient. Preferably, a vaccine contains from about from about 1×10⁸ to about 5.5×10¹⁰ myeloid cells per single dose per patient, and in another embodiment, from about 1×10⁶ to about 20×10⁶ myeloid cells per single dose per patient, and in another embodiment, from about 1×10⁶ to about 10×10⁶ myeloid cells per single dose per patient. These doses are given for a typical human or other primate. Doses suitable for other animals can be determined by those of skill in the art. For example, for a mouse, a suitable dose is from about 1×10⁶ to about 3×10⁶ per single dose per mouse. Other doses can be determined by the skilled artisan and is well within the ability of those of skill in the art. “Boosters” of a vaccine are preferably administered when the immune response against the antigen has waned or as needed to provide an immune response or induce a memory response against a particular antigen or antigen(s). Boosters can be administered from about 2 weeks to several years after the original administration.

A vaccine of the present invention can be formulated with various pharmaceutically acceptable adjuvants, carriers and/or excipients. Adjuvants are typically substances that generally enhance the immune response of an animal to a specific antigen. Suitable adjuvants include, but are not limited to, Freund's adjuvant; other bacterial cell wall components; aluminum-based salts; calcium-based salts; silica; polynucleotides; toxoids; serum proteins; viral coat proteins; other bacterial-derived preparations; gamma interferon; block copolymer adjuvants, such as Hunter's Titermax adjuvant (CytRx™, Inc. Norcross, Ga.); Ribi adjuvants (available from Ribi ImmunoChem Research, Inc., Hamilton, Mont.); and saponins and their derivatives, such as Quil A (available from Superfos Biosector A/S, Denmark).

Carriers are typically compounds that increase the half-life of a therapeutic composition in the treated animal. Suitable carriers include, but are not limited to, polymeric controlled release formulations, biodegradable implants, liposomes, oils, esters, and glycols.

Vaccines and therapeutic compositions of the present invention can also contain one or more pharmaceutically acceptable excipients. As used herein, a pharmaceutically acceptable excipient refers to any substance suitable for delivering a therapeutic composition useful in the method of the present invention to a suitable in vivo or ex vivo site. Preferred pharmaceutically acceptable excipients are capable of maintaining a vaccine in a form that, upon arrival of the vaccine at a target cell, tissue, or site in the body, the vaccine is capable of eliciting an immune response at the target site (noting that the target site can be systemic). Suitable excipients of the present invention include excipients or formularies that transport, but do not specifically target the vaccine to a site (also referred to herein as non-targeting carriers). Examples of pharmaceutically acceptable excipients include, but are not limited to water, saline, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol.

Therefore, the present invention includes the delivery of isolated myeloid cells (and/or progenitors or progeny thereof) of the present invention (including vaccines/compositions comprising such cells) to an animal. The isolated myeloid cell used in the treatment can be produced in vitro, or isolated from the patient and then activated ex vivo before administering the cell to the patient. In one embodiment, the invention includes a vaccine that comprises direct progenitors of the myeloid cell of the invention and/or earlier progenitors such as earlier myeloid precursor cells, stem cells, or bone marrow cells, and the vaccine further comprises an agent that causes precursor cells to differentiate or become activated to produce the myeloid cells of the invention. Such a vaccine can also include an antigen or biological response modifier. In this manner, the cells can be differentiated and/or activated after administration of the vaccine, in vivo.

According to the present invention, ex vivo administration refers to performing part of the regulatory step outside of the patient, such producing and activating the myeloid cells from bone marrow that was removed from a patient or activating a myeloid cell line of the invention, and returning the activated cells to the patient. The vaccine according to the present invention can be administered to a patient by any suitable mode of administration. Such administration can be systemic, mucosal and/or proximal to the location of a target site. The preferred routes of administration will be apparent to those of skill in the art, depending on the type of condition to be prevented or treated. Preferred methods of administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, intraspinal, pulmonary administration, impregnation of a catheter, and direct injection into a tissue.

According to the present invention, an effective administration protocol comprises suitable dose parameters and modes of administration that result in delivery of a useful number of functional myeloid cells of the present invention to a patient in order to provide a transient or long term benefit to the patient. Effective dose parameters can be determined using methods standard in the art for a particular condition or disease. Such methods include, for example, determination of survival rates, side effects (i.e., toxicity) and progression or regression of disease.

As used herein, the phrase “protected from a disease” refers to reducing the symptoms of the disease; reducing the occurrence of the disease, and/or reducing the severity of the disease. Protecting an animal can refer to the ability of myeloid cells of the present invention, when administered to an animal, alone or in conjunction with other components (antigen, biological response modifiers, etc.), to prevent a disease from occurring and/or to cure or to alleviate disease symptoms, signs or causes. As such, to protect an animal from a disease includes both preventing disease occurrence (prophylactic treatment) and treating an animal that has a disease or that is experiencing initial symptoms of a disease (therapeutic treatment). The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

In one embodiment, the present invention includes a method for enhancing a thymus-dependent immune response, comprising: (a) isolating the myeloid cell (and/or progenitors thereof) as described herein from a patient; (b) activating the cell ex vivo; and (c) administering the cell after step (b) to the patient. In one aspect, the method further includes administering an antigen to the patient. In part (b), the step of activating can include exposing the cell to an agent selected from the alum and GM-CSF, or a derivative thereof. The myeloid cell of the present invention, as previously described herein, can be isolated from any suitable tissue, including, but not limited to, the bone marrow, the spleen, or the peripheral blood of the patient.

In another embodiment, the present invention includes a method for enhancing a thymus-dependent immune response, comprising: (a) providing a myeloid cell according to the present invention (and/or progenitors thereof); (b) activating the cell ex vivo; and (c) administering the cell after step (b) to the patient. In one aspect, the method further includes administering an antigen to the patient. Step (b) of activating has been discussed above. Also as discussed above, the step of providing can include any means of providing including, but not limited to, producing the cell as described herein or obtaining the cell from another source (e.g., by purchasing the cell or obtaining the cell from another laboratory or individual).

Other aspects of the methods of the invention (e.g., administration with biological response modifiers, adjuvants, etc.) have been described in detail above. For example any of the above-described methods can be used to enhance an IL-4 associated immune response or equivalent thereof, a thymus-dependent immune response, or more particularly, to enhance B cell priming of MHC Class II signaling, B cell expansion and/or B cell antibody production.

In the method of the present invention, myeloid cells produced or isolated according to the method of the invention, and compositions comprising the myeloid cells or progenitors thereof can be administered to any animal, including any member of the Vertebrate class, Mammalia, including, without limitation, primates, rodents, livestock and domestic pets. A preferred mammal to treat is a human. The term “patient” as used herein, unless specifically defined, can refer to any animal that is treated or contacted using the present invention.

Another embodiment of the present invention relates to a method to identify adjuvants and other agents/compounds that enhance immune responses, including, but not limited to, thymus-dependent immune responses, IL-4 associated immune responses (or equivalent immune responses), B cell priming of MHC Class II signaling, B cell expansion and/or B cell antibody production. The method includes the steps of: (a) exposing a source of progenitors of the myeloid cell of the present invention to a test adjuvant or compound; (b) detecting cells from (a) that, after exposure to the test adjuvant, have the following phenotype: CD11b⁺, CD11c^{-/low}, MHC Class^{II-}, or the phenotype of the myeloid cell of the present invention; and (c) determining whether cells detected in (b), when contacted with naive B cells, mediate priming of B cells for MHC class II signaling, mediate IL-4 associate immune responses, mediate thymus-dependent immune responses, mediate B cell expansion, and/or mediate B cell antibody production. An induction or increase in any of these functional characteristics of a myeloid cell of the present invention when the cells are exposed to the adjuvant indicates that the adjuvant is useful for enhancing such responses and more particularly, for activating a myeloid cell of the invention. The step (a) of the method can, in one aspect, be performed in vivo by administering the test adjuvant to an animal and isolating bone marrow cells, adult stem cells, or spleen cells from the animal prior to performing step (b). In one embodiment, the test adjuvant or agent is administered together with an antigen. In another embodiment, step (a) is performed in vitro by exposing the cells to the test adjuvant or agent in a culture.

The conditions under which the cells are exposed to or contacted with a test agent, such as by mixing, or by in vivo administration, are any suitable culture or assay conditions or in vivo administration conditions, respectively. In the case of an in vitro assay, the step includes an effective medium in which the cell can be cultured and evaluated in the presence and absence of the test agent. Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art and are shown in the Examples section. Methods to evaluate the expression of cell surface markers and biological activity of the myeloid cells have been discussed above and are shown in the Examples section.

The following examples are provided for the purpose of illustration and are not intended to limit the scope of the present invention.

### EXAMPLES

### Example 1

The following example demonstrates that adjuvant-induced signals prime MHC class II/Ig-α/β signaling in a large portion of splenic B cells.

To assess whether priming for MHC class II-mediated Ca²⁺ mobilization was specifically associated with immune responses, the present inventors analyzed the ability of B cells to mobilize intracellular Ca²⁺ in response to MHC class II aggregation following immunization with nitrophenyl (NP)-conjugated bovine serum albumin (BSA) precipitated in aluminum hydroxide (alum) (8). Alum is the most widely used adjuvant for human vaccines, and has been used experimentally since the 1920's to promote antibody responses to vaccination in animals (9). Although adsorbtion of antigen onto particulate alum is thought to produce an important depot-effect, the precise mechanisms by which alum promotes immune responses remain unclear.

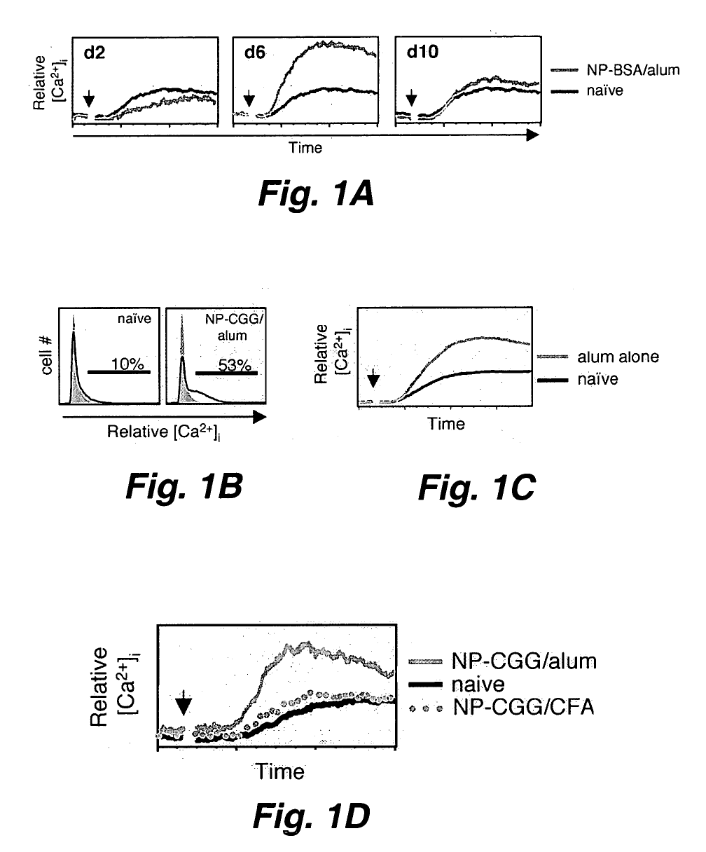

Briefly, C57/BL6 mice were injected with 200 μg NP-BSA/alum (FIG. 1A), alum alone (FIG. 1C), NP-CGG/alum (FIGS. 1B and 1D), or NP-CGG/CFA (FIG. 1D). MHC class II-mediated Ca²⁺ mobilization was analyzed on: days 2, 6, and 10 (FIG. 1A) or day 6 post-injection (FIGS. 1B, 1C, 1D). The percentage of B cells mobilizing intracellular Ca²⁺ following MHC class II aggregation shown in FIG. 1B was determined by comparison of intracellular Ca²⁺ levels in resting cells (shaded histograms) with intracellular free Ca²⁺ levels 3 minutes following MHC class II aggregation (black line histograms). All data shown in FIGS. 1A-1D represent B220⁺ populations only. Arrows indicate MHC class II aggregation. Results are representative of at least three independent experiments.

Using this immunization method, maximal priming of splenic B cells was observed in vivo at 6 days. It was no longer apparent by 10 days post-immunization (FIG. 1A). Priming occurred in over 50% of B cells from immunized mice (FIG. 1B) and could be achieved by injection of precipitated alum alone (FIG. 1C), revealing that the process was not antigen-dependent. Unexpectedly, this effect was not observed following challenge with complete Freund's adjuvant (CFA) (FIG. 1D), another classic vaccine adjuvant used in animals. Although alum administration alone was sufficient for in vivo priming, for consistency, subsequent analyses in the Examples below used antigen/alum precipitates.

### Example 2

The following example demonstrates that Gr1⁺ myeloid cells accumulate in the spleens of immunized mice and are required for in vivo priming of MHC class II/Ig-α/β signaling in B cells.

Based on the antigen independence of the B cell priming shown in Example 1, the present inventors reasoned that the phenomenon might be induced by cells responding to innate immune signals provided by alum. Therefore, non-lymphoid cell populations in the spleens of mice were analyzed 6 days after administration of NP-BSA/alum. Briefly, flow cytometric analysis of the various splenic populations discussed below was conducted on day 6 following exposure of C57/BL6 mice to NP-BSA/alum or alum alone. Contrary to the inventors' expectations, no significant increases in cells expressing CD11c⁺ were detected (data not shown). However, an accumulation of cells displaying high levels of the surface markers, CD11b and Gr1, was observed (data not shown). While these cells accumulated in the spleen after intraperitoneal vaccination, a similar though less robust splenic accumulation occurred after subcutaneous administration of alum, a route which is more similar to that used for human vaccination (10). Interestingly, injection with alum alone or with NP-BSA/CFA led to comparable increases in Gr1⁺/CD11b⁺ cells in the spleen (data not shown), despite the fact that B cells were not primed after CFA administration. The accumulating Gr1⁺ population was also CD11c^{-/low}, MHC class II⁻, F4/80⁺ and CD68^{+/-} (data not shown). Despite expression of the granulocytic marker Gr1, co-expression of the monocyte/macrophage lineage markers CD68 and F4/80 by many of these cells suggested that they were not neutrophils.

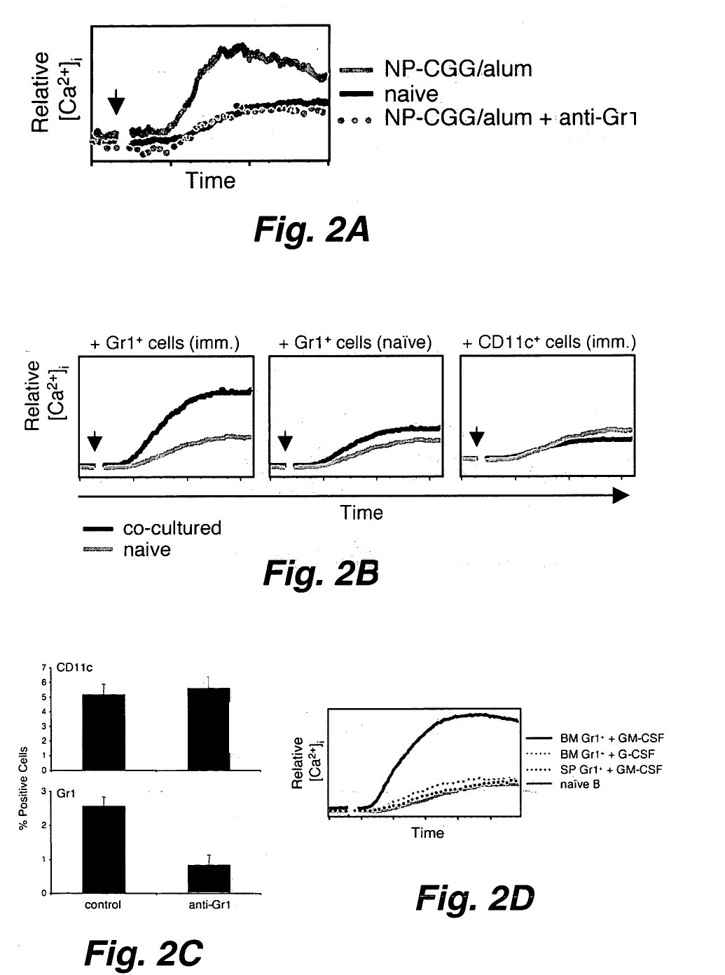

To investigate whether the Gr1⁺ cell population might participate in the induction of MHC class II signaling, Gr1⁺ cells were selectively depleted after injection of mice with alum (FIG. 2A and FIG. 2C) (8). Briefly, MHC class II-mediated Ca²⁺ mobilization in splenic B220+cells was analyzed on day 6 following exposure of C57/BL6 mice to NP-BSA/alum with or without administration of depleting anti-Gr1⁺ antibody. This in vivo depletion of Gr1⁺ cells completely abrogated alum-induced priming (FIG. 2A), demonstrating that alum specifically induces the accumulation of a myeloid cell population that is required for priming of MHC class II/Ig-α/β signaling in B cells. Referring to FIGS. 2A and 2B, the arrows indicate MHC class II aggregation. Images are representative of three independent analyses.

To examine whether the alum-induced Gr1⁺ cells acted directly on B cells, Gr1⁺ cells were sorted from the spleens of alum-treated mice and co-cultured with naïve B cells. Briefly, MHC class II-mediated Ca²⁺ mobilization by B cells was analyzed after coculture for 18 hours with Gr1⁺ or CD11c⁺ cells which were sorted from alum-injected or naïve mice in separate experiments. Splenic Gr1⁺ cells from alum-exposed mice were able to prime MHC class II-mediated Ca²⁺ mobilization in B cells (FIG. 2B) (8). This effect was restricted to Gr1⁺ cells from alum-exposed mice, since neither CD11c+spleen cells from the same animals nor splenic Gr1⁺ cells from resting mice could produce the same effects. In addition, culture of bone marrow Gr1⁺ cells with granulocyte-macrophage colony-stimulating factor (GM-CSF) (but not granulocyte colony-stimulating factor (G-CSF)) induced similar effects to in vivo alum administration by rendering these myeloid cells capable of priming B cells upon co-culture (FIG. 2D).

Immunofluorescence analysis of spleen sections revealed that while many Gr1⁺ cells are found in the red pulp, a significant number are found intimately associated with IgM^{bright} B cells in periarteriolar lymphatic sheath-associated foci. Thus, some of these cells are optimally situated for direct interactions with B cells (data not shown).

### Example 3

The following example demonstrates that Gr1⁺ cells are associated with IL-4, which is required for priming of MHC class II/Ig-α/β signaling in B cells.

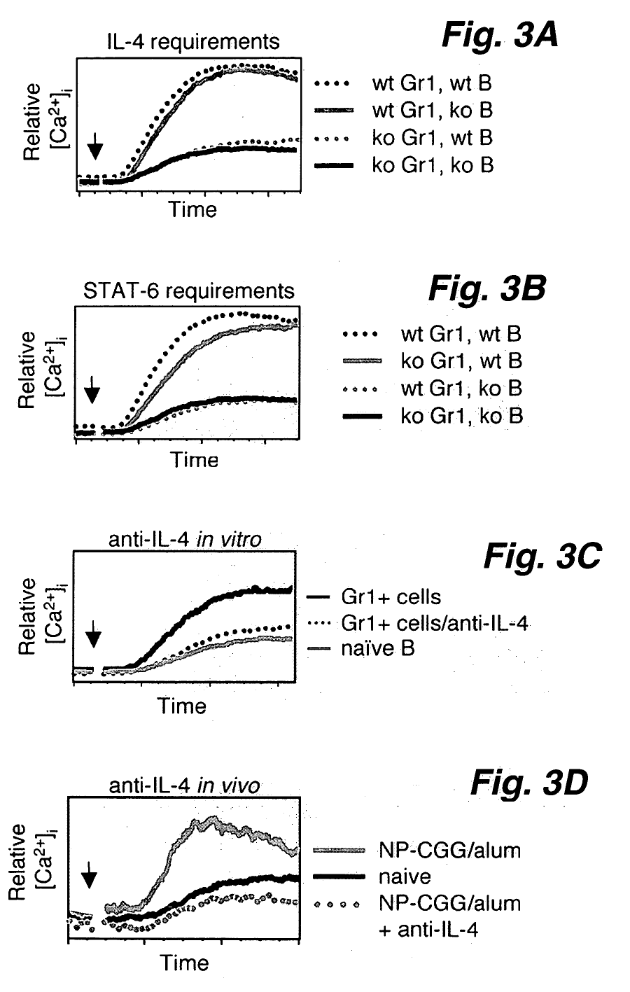

Next, the mechanism by which Gr1⁺ myeloid cells prime B cells was explored. Since IL-4 is the only reported polyclonal activator of this form of priming, the inventors tested the involvement of this cytokine by the addition of IL-4 neutralizing antibody to co-cultures containing Gr1⁺ cells from spleens of alum challenged mice and naïve B cells. First, MHC class II-mediated Ca²⁺ mobilization was assessed in B cells following 18 hour co-cultures with Gr1⁺ cells sorted from the spleens of NP-BSA/alum-immunized mice in the presence or absence of blocking anti-IL-4 antibodies. Results showed that blocking IL-4 completely abolished B cell priming (FIG. 3C).

In order to clarify which cells were producing IL-4 and whether the IL-4 acted directly on B cells, B cells were cultured with GM-CSF-activated Gr1⁺ bone marrow cells taken from wild type, IL-4 deficient, or STAT-6 deficient mice. Briefly, GM-CSF-activated bone marrow derived Gr1⁺ cells and naïve B cells from wild type, IL-4^{-/-}, or STAT6^{-/-} mice were co-cultured for 18 hours and MHC class 11-mediated Ca²⁺ mobilization was analyzed. The results demonstrated that Gr1⁺ cell-derived IL-4 and B cell expression of STAT-6 are required for B cell priming (FIGS. 3A and 3B). Since STAT-6 is required for most responses mediated by the IL-4 receptor (11), these data indicate that Gr1⁺ cell-derived IL-4 was acting directly on B cells to produce priming.

Finally, MHC class II-mediated Ca²⁺ mobilization was assessed in splenic B cells on day 6 post-exposure to NP-BSA/alum alone or in conjunction with blocking anti-IL-4 antibodies (1 mg given IV on days 3 and 5 post-immunization). Results show that, consistent with these in vitro observation, administration of IL-4 neutralizing antibodies following NP-BSA/alum challenge completely blocked in vivo priming of splenic B cells (FIG. 3D).

To examine the presence of IL-4 expressing Gr1+ cells in alum challenged mice, IL-4 reporter mice were used in which GFP expression is under the control of the IL-4 promoter (12). Because alum was able to prime MHC class II signaling in vivo, while CFA was not, the ability of these two adjuvants to induce GFP expression in various splenic populations was compared. Briefly, flow cytometric analysis of splenic cells from IL-4 reporter mice was performed 6 days post-exposure to NP-BSA/alum, NP-BSA/CFA, or nothing. The results showed that 6 days after administration of alum, a splenic GFP⁺ cell population was apparent, most of which were Gr1⁺, CD11b⁺, and F4/80⁺ (data not shown and (10)). However, only about 25% of the total Gr1⁺ cell population in the spleen was also GFP+. In contrast, while CFA induced a similar accumulation of Gr1⁺ cells in the spleen, these cells did not express GFP. It was confirmed that the Gr1⁺/GFP⁺ cells were producing IL-4 by sorting them and assaying culture supernatants for IL-4 by enzyme-linked immunosorbant assay (ELISA) (25 U/ml for GFP⁺ and 0 U/ml for GFP⁻).

Next, Gr1⁺, F4/80⁺ and Gr1⁺, F4/80⁻ cells were sorted from the spleens of wild type mice 6 days post-exposure to NP-BSA/alum and stained with hemotoxylin and eosin to assess morphology. More than half of sorted Gr1⁺, F4/80⁺ cells from the spleens of alum challenged mice exhibited a mononuclear morphology, while Gr1⁺, F4/80⁻ cells uniformly displayed the typical polymorphonuclear morphology of neutrophils (data not shown and (10)).

Taken together, these data indicate that Gr1⁺ cells accumulate in the spleen following challenge with either alum or CFA, but that a subset selectively produces IL-4 after alum administration. Furthermore, these IL-4 producing cells are largely of the monocyte/macrophage lineage. Recently, Geissmann et al have demonstrated that an inflammatory subset of murine monocytes express Gr1(13). The IL-4⁺/GR1⁺ cells seen by the present inventors after vaccination appear to be quite similar cells. They do not appear to be plasmacytoid dendritic cells, however, because they are B220⁻, MHC class II⁻, and largely CD11c⁻ (10, 14).

### Example 4

The following example demonstrates that Gr1⁺ cells facilitate B cell responses following thymus dependent antigenic challenge.

In the next experiment, the relative importance of alum-induced Gr1⁺ cells for effective responses to thymus-dependent antigens was determined. GFP transgenic B cells which were specific for NP (B1-8/GFP) (15) were transferred into naïve wild-type mice, and the recipients were then immunized with NP-OVA/alum and then administered either anti-Gr1 or control antibodies. More specifically, 1×10⁷ purified NP-specific B cells from Ig-knockin B1-8X GFP mice were transferred intravenously into syngeneic recipients, which were subsequently immunized with NP-OVA/alum. Recipients also received depleting anti-Gr1 or control antibodies. Expansion of transferred NP-specific cells was monitored by analyzing the percentage of GFP⁺/B220⁺ cells in spleens. Examination of recipient spleens six days later revealed that expansion of transferred B cells was substantially reduced by administration of anti-Gr1 antibody (data not shown).

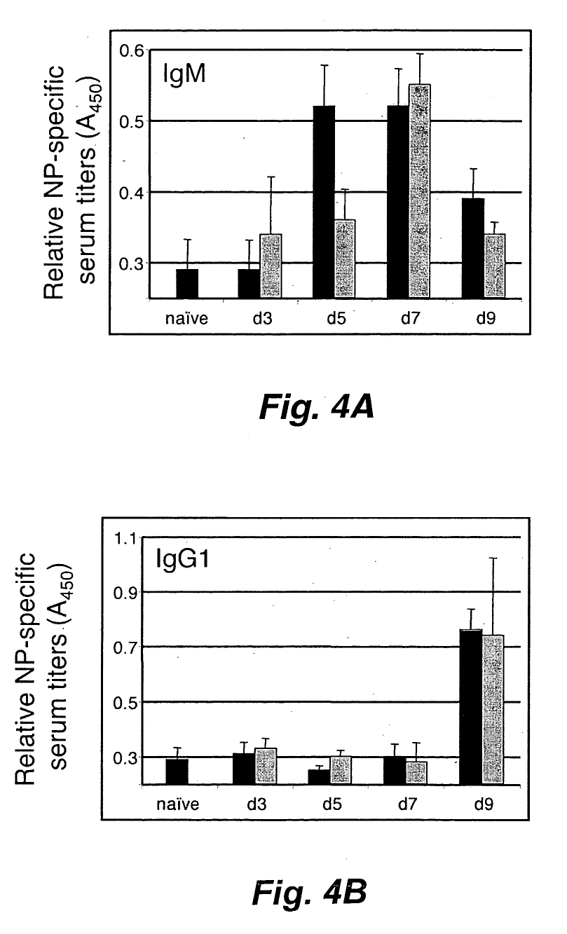

Next, naïve wild-type mice were vaccinated with NP-OVA/alum and administered either control or anti-Gr1 antibody. More specifically, C57/BL6 mice were vaccinated with NP-OVA/alum (10 μg) and subsequently treated with either anti-Gr1 or control antibodies. Sera were assayed for NP-specific antibody by ELISA. The results showed that primary anti-NP IgM responses were compromised by treatment with anti-Gr1 antibody (FIGS. 4A and 4B). Thus, Gr1⁺ cells are important for the expansion of antigen-specific B cell populations and optimal thymus-dependent antibody responses.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. An isolated myeloid cell and progenitors and progeny thereof, wherein the cell expresses CD11b, wherein the cell does not express MHC Class II, and wherein the cell expresses low levels of or does not express CD11c.

2. The isolated myeloid cell of claim 1, wherein the cell expresses F4/80.

3. The isolated myeloid cell of claim 1, wherein the cell expresses CD68.

4. The isolated myeloid cell of claim 1, wherein the cell expresses CCR3.

5. The isolated myeloid cell of claim 1, wherein the cell expresses B220.

6. The isolated myeloid cell of claim 1, wherein the cell does not stain with vital red stain.

7. The isolated myeloid cell of claim 1, wherein the cell does not express CD86.

8. The isolated myeloid cell of claim 1, wherein the cell does not express a T cell receptor (TcR) or a surface immunoglobulin.

9. The isolated myeloid cell of claim 1, wherein the cell is a murine cell and wherein the cell expresses Gr1.

10. The isolated myeloid cell of claim 1, wherein the cell is a human cell and wherein the cell expresses the human homologue of murine Gr1.

11. The isolated myeloid cell of claim 1, wherein the cell, when activated, mediates an immune response.

12. The isolated myeloid cell of claim 1, wherein the cell, when activated, mediates an immune response associated with IL-4 production.

13. The isolated myeloid cell of claim 1, wherein the isolated myeloid cell, when activated, mediates priming of B cells for MHC class II signaling.

14. The isolated myeloid cell of claim 1, wherein the cell mediates thymus-dependent B cell expansion.

15. The isolated myeloid cell of claim 1, wherein the cell mediates thymus-dependent antibody production by B cells.

16. The isolated myeloid cell of claim 1, wherein the isolated myeloid cell is activated by an aluminum-based salt adjuvant.

17. The isolated myeloid cell of claim 1, wherein the isolated myeloid cell is activated by granulocyte-macrophage colony-stimulating factor (GM-CSF).

18. The isolated myeloid cell of claim 1, wherein the cell is derived from a cell isolated from bone marrow that has been exposed to granulocyte-macrophage colony-stimulating factor (GM-CSF).

19. The isolated myeloid cell of claim 1, wherein the cell is derived from a cell isolated from bone marrow that has been contacted with an aluminum-based salt adjuvant.

20. The isolated myeloid cell of claim 1, wherein the cell has been immortalized.

21. An isolated population of cells enriched for the isolated myeloid cell or progenitors or progeny thereof of claim 1.

22. The isolated population of cell of claim 22, wherein the population is a clonal population consisting essentially of the myeloid cell and progeny thereof of claim 1.

23. The isolated population of cells of claim 22, wherein the population of cells is produced by:
a) isolating cells from a source selected from the group consisting of: bone marrow, hematopoietic precursor cells, adult stem cells, fetal stem cells, spleen cells, peripheral blood cells and embryonic stem cells;
b) exposing the cells to an agent selected from the group consisting of an aluminum-based salt adjuvant and GM-CSF, or a derivative thereof;
c) isolating cells from step (b) that have the following cell surface phenotype: CD11b⁺, CD11c^{-/low}, MHC Class^{II-}.

24. The isolated population of cells of claim 22, wherein the population of cells is produced by:
a) immunizing an animal with a composition comprising an aluminum-based salt adjuvant or a derivative thereof;
b) isolating cells from step (a) that have the following cell surface phenotype: CD11b⁺, CD11c^{-/low}, MHC Class^{II-}.

25. A vaccine comprising the isolated myeloid cell or its progenitor of claim 1 and at least one antigen.

26. The vaccine of claim 25, wherein the antigen is selected from the group consisting of: a viral antigen, a mammalian cell surface molecule, a bacterial antigen, a fungal antigen, a protozoan antigen, a helminth antigen, an ectoparasite antigen, and a cancer antigen.

27. A vaccine comprising the isolated myeloid cell or its progenitor of claim 1 and a cytokine.

28. A method for enhancing a thymus-dependent immune response, comprising:
a) isolating the myeloid cell or its progenitor of claim 1 from a patient;
b) activating the cell ex vivo; and
c) administering the cell after step (b) to the patient.

29. The method of claim 28, wherein step (c) further comprises administering an antigen to the patient.

30. The method of claim 28, wherein step (b) comprises exposing the cell to an agent selected from the group consisting of an aluminum-based salt adjuvant and GM-CSF.

31. The method of claim 28, wherein the myeloid cell in (a) is isolated from the bone marrow, the spleen, or the peripheral blood of the patient.

32. A method for enhancing a thymus-dependent immune response, comprising:
a) providing a myeloid cell or its progenitor according to claim 1;
b) activating the cell ex vivo; and
c) administering the cell after step (b) to the patient.

33. The method of claim 32, wherein step (c) further comprises administering an antigen to the patient.

34. The method of claim 32, wherein step (b) comprises exposing the cell to an agent selected from the group consisting of an aluminum-based salt adjuvant and GM-CSF.

35. A method to produce a myeloid cell that mediates thymus-dependent immune responses, comprising:
a) isolating cells from the bone marrow, spleen or peripheral blood of an animal;
b) exposing the cells to an agent selected from the group consisting of an aluminum-based salt adjuvant and GM-CSF, or a derivative thereof; and
c) selecting cells from (b) that have the following cell surface phenotype: CD11b⁺, CD11c^{-/low}, MHC Class^{II-}.

36. The method of claim 35, wherein the agent in step (b) is selected from the group consisting of an aluminum-based salt adjuvant and GM-CSF.

37. A method to identify agents that enhance thymus-dependent immune responses, comprising:
a) exposing a source of myeloid progenitor cells to a test agent;
b) detecting whether cells from (a) that, after exposure to the test adjuvant, comprise cells having the following phenotype: CD11b⁺, CD11c^{-/low}, MHC Class^{II-}; and
c) determining whether cells detected in (b), when contacted with naive B cells, mediate priming of B cells for MHC class II signaling;
wherein an induction or increase in priming of B cells for MHC class II signaling when the bone marrow cells are exposed to the adjuvant indicates that the adjuvant is useful for enhancing thymus-dependent immune responses.

38. The method of claim 36, wherein step (a) is performed in vivo by administering the test adjuvant to an animal and isolating bone marrow cells, stem cells, or spleen cells from the animal prior to performing step (b).

39. The method of claim 38, wherein the test adjuvant is administered together with an antigen.

40. The method of claim 37, wherein step (a) is performed in vitro by exposing the cells to the test adjuvant in a culture.

41. The method of claim 37, wherein the myeloid progenitor cells are selected from the group consisting of: bone marrow cells, adult stem cells, fetal stem cells, embryonic stem cells, hematopoietic precursor cells, spleen cells, peripheral blood cells, a direct progenitor of the myeloid cell according to claim 1.
